(19) United States
(12) Patent Application Publication
(10) Pub. No.: US 2006/0269934 A1
(43) Pub. Date: Nov. 30, 2006

(54) Compositions and methods for clonal amplification and analysis of polynucleotides

(75) Inventors: Timothy M. Woudenberg, Moss Beach, CA (US); Cheryl Heiner, La Honda, CA (US); Eric S. Nordman, Palo Alto, CA (US)

Correspondence Address: DECHERT LLP, P.O. BOX 10004, PALO ALTO, CA, 94303, US

(73) Assignee: Applera Corporation, Foster City, CA (US)

(21) Appl. No.: 11/377,763

(22) Filed: Mar. 16, 2006

Related U.S. Application Data
(60) Provisional application No. 60/662,961, filed on Mar. 16, 2005.

(51) Int. Cl. C12Q 1/68; C12P 19/34; C12M 1/34

(52) U.S. Cl. 435/6; 435/91.2; 435/287.2

(57) ABSTRACT

Compositions and methods of use are disclosed for clonally amplifying and analyzing one or more polynucleotides.

### 1. CROSS REFERENCE TO RELATED APPLICATIONS

This application claims benefit under 35 U.S.C. § 119(e) to application Ser. No. 60/662,961 filed Mar. 16, 2005, the contents of which are incorporated herein by reference.

### 2. BACKGROUND

Current methods for routine and large scale analysis of target polynucleotides require the preparation of tens to thousands to millions of individual target samples, which are processed and analyzed in individual reaction vessels. Therefore, labor, materials, and equipment make up a significant cost of any target polynucleotide analysis regardless of the methodology employed.

To increase the number of target polynucleotides that can be discretely and simultaneously analyzed, the present disclosure includes compositions and methods for clonally amplifying target polynucleotide sequences and the parallel analysis of the amplified sequences.

### 3. SUMMARY

These and other features of the present teachings are set forth herein.

This disclosure provides compositions, methods, and kits for the analysis of polynucleotides. In general, the disclosure provides methods of isolating and clonally amplifying polynucleotides to produce isolated populations of amplicons (“clonal amplicons”). In some embodiments, very large numbers of polynucleotides can be isolated and clonally amplified in parallel.

Polynucleotides can be isolated and clonally amplified by various methods. In some embodiments, polynucleotides can be isolated and clonally amplified by insertion into recombinant vectors, which can be introduced into a host cell suitable for replicating the vector. Polynucleotides also can be isolated and clonally amplified and placed in a reaction vessel or in a hydrophilic compartment of an inverse emulsion.

Various methods or techniques can be used to clonally amplify isolated polynucleotides, such as, PCR, including exponential, linear, log-linear, and asymmetric PCR. Therefore, in some embodiments, clonal amplification reactions can include one or more primers. In some embodiments, a primer used in a clonal amplification reaction can be attached to a surface, such as, a microparticle, bead or a slide. In some embodiments, a surface can comprise a plurality of primers. Therefore, in various exemplary embodiments, the products of clonal amplification (i.e., isolated populations of clonal amplicons) can be attached to a surface.

In some embodiments, a primer attached to a surface can be used to clonally amplify a polynucleotide isolated in a hydrophilic compartment of an inverse emulsion. In some embodiments, (e.g., when a primer is attached to a microparticle) the entire surface of the microparticle can be completely contained within the hydrophilic compartment. In some embodiments, (e.g., when a primer is attached to a slide) the hydrophilic compartment can be disposed upon the slide, and therefore, may not be completely contained within the hydrophilic compartment. In some embodiments, a surface to which a primer is a attached can be external to the hydrophilic compartment.

In some embodiments, polynucleotides can be selected for analysis to the exclusion of other polynucleotides or polynucleotide sequences that can be present in sample. For example, in some embodiments, polynucleotides can be selected based on their suitability for incorporation into a recombinant vector and/or their suitability for replication by various host cells.

In some embodiments, polynucleotides can be selected using a multiplex amplification reaction. In some embodiments, a multiplex amplification reaction is suitable to select and amplify hundreds, thousands, hundreds of thousands, or millions of polynucleotides to produce multiplex amplicons, that can be isolated and clonally amplified.

Once made, the clonal amplicons can be analyzed by various methods. In some embodiments, the methods of analysis can be suitable for analyzing various populations of isolated clonal amplicons in parallel. The number of clonal amplicons that can be analyzed in parallel can be determined at the discretion of the practitioner and can include hundreds, thousands, hundreds of thousands, or millions of clonal amplicons. The methods of analysis include but are not limited to detection, single nucleotide polymorphism analysis, sequencing and the like. In various exemplary embodiments, sequencing can be by parallel sequencing, pyrosequencing, fluorescence in situ sequencing, or massively parallel signature sequencing.

### 4. BRIEF DESCRIPTION OF THE FIGURES

The skilled artisan will understand that the drawings, described below, are for illustration purposes only. The drawings are not intended to limit the scope of the present teachings in any way.

FIG. 1 illustrates an embodiment of multiplex amplification of a transcriptome 1 by polymerase chain reaction (PCR). Each forward 10 and reverse primer 20 pair contains a forward 30 and reverse 40 universal sequences that are incorporated into each amplicon 50.

FIG. 2 illustrates an embodiment of an inverse emulsion 120 comprising a hydrophobic phase 125 and a plurality of hydrophilic compartments 130A-D. Hydrophilic compartment 130A contains isolated polynucleotide 200, multiple copies of reverse primer 80, and multiple copies of forward primer 100 attached to microparticle 90. Clonal amplification of polynucleotide 200 by PCR yields an isolated population of double stranded clonal amplicons 105 attached to microparticle 90.

FIG. 3 illustrates an embodiments of an inverse emulsion 150 comprising a plurality of hydrophilic compartments 140A-B disposed upon surface 110. Compartment 140A contains isolated target polynucleotide 60, multiple copies of reverse primer 80, and multiple copies of forward primer 100 attached to surface 110.

FIG. 4 illustrates an embodiment of clonal amplicons 200, 300, 400 attached to discrete areas of a surface 110.

### 5. DETAILED DESCRIPTION

It is to be understood that both the foregoing general description, including the drawings, and the following detailed description are exemplary and explanatory only and are not restrictive of this disclosure. In this disclosure, the use of the singular includes the plural unless specifically stated otherwise. Also, the use of “or” means “and/or” unless stated otherwise. Similarly, “comprise,” “comprises,” “comprising” “include,” “includes,” and “including” are not intended to be limiting.

This disclosure provides compositions, methods, and kits for the analysis of polynucleotides. In general, the disclosure provides methods of isolating and amplifying polynucleotides under conditions suitable for producing isolated populations of amplified sequences. “Isolated” as used herein refers to placed or standing alone, discrete, detached, separated from others. Therefore, “isolated polynucleotide” as used herein refers to a polynucleotide that is detached or separated from other polynucleotides in a manner and under conditions suitable to yield isolated groups or populations of amplified sequences (“amplicons”). The amplicons that comprise any given isolated population can be traced directly or indirectly to an isolated polynucleotide and can be referred to as “amplicon clones” or “clonal amplicons”. Therefore, the disclosed methods of isolating and amplifying polynucleotides to yield isolated populations of amplicons can be referred to as “clonal amplification”. The methods and techniques employed in the analysis of clonal amplicons can be selected at the discretion of the practitioner and include but are not limited to detection, sequencing, resequencing, quantitation, single-nucleotide polymorphism analysis, and the like.

The methods disclosed herein are suitable for analyzing complex polynucleotides and complex mixtures of polynucleotides. For example, in some embodiments, the disclosed methods can be used to sequence an entire genome of a cell, organism, or virus. However, in some embodiments, the methods disclosed herein can be used to select a subset of specific polynucleotides sequences of a genome for analysis. Therefore, in some embodiments, specific polynucleotide sequences of interest can be selected, clonally amplified, and analyzed to the exclusion of other polynucleotide sequences that may be present in a sample. As described in more detail below, various methods and techniques can be used to select polynucleotides of interest. These include but are not limited to amplification techniques (e.g., PCR techniques), hybridization techniques insertion of polynucleotides of interest into a recombinant vector, and the like. The methods disclosed herein are suitable for clonal amplification and analysis of a plurality of polynucleotides. In some embodiments, a plurality of polynucleotides can be clonally amplified and analyzed in parallel. “Parallel reaction” as used herein refers to a reaction solution comprising a plurality of discrete regions suitable for performing a plurality of reactions concurrently. In some embodiments, the discrete regions of a parallel reaction can be in fluidic communication. Therefore, in some embodiments, reactants and/or products can be exchanged between the various discrete regions. However, in general, certain reactants, including but not limited to, polynucleotides and clonal amplicons are retained within discrete regions of a parallel reaction to facilitate their analysis by the methods disclosed herein. Virtually any number of polynucleotides can be clonally amplified and analyzed. For example, in various exemplary embodiments, hundreds, thousands, hundreds of thousands millions, and even greater numbers of polynucleotides can be analyzed in parallel by the disclosed methods. In various exemplary embodiments the numbers of polynucleotides analyzed in parallel can be at least 100, 500, 1000, 10000, 50000, 100000, 300000, 500000, or 1000000.

In some embodiments, limiting dilution can be used to isolate polynucleotides in a manner that is suitable for clonal amplification. For example, a sample comprising a plurality of polynucleotides can be diluted to a concentration such that aliquots of the diluted sample that can be placed into individual reaction vessels (e.g., wells of a multi-well plate) can be predicted to comprise on average >0 and <1 polynucleotide. Therefore, a percentage of reaction vessels can be predicted on a statistical basis (e.g., Poisson distribution) to comprise an isolated polynucleotide suitable for clonal amplification. Determining a dilution suitable for obtaining isolated polynucleotides is within the abilities of the skilled artisan. Factors to be considered include but are not limited to the polynucleotide concentration and the expected number, types, and composition of various polynucleotides that may be present in a sample. In some embodiments, a dilution suitable for obtaining isolated polynucleotides from a sample can be determined empirically. Once isolated within the reaction vessels, polynucleotides can be amplified by various methods as described below to produce clonal amplicons.

In some embodiments, a semi-solid or gel matrix can be used to isolate polynucleotides in a manner suitable for clonal amplification. In some embodiments, this can be accomplished by mixing polynucleotides at a suitable concentration with a matrix-forming material and allowing the material to set (e.g., agarose, acrylamide, etc.) The composition and consistency of the matrix can be selected at the discretion of the practitioner. However, in some embodiments, the matrix can be suitable for retaining polynucleotides and amplified sequences at discrete locations within the matrix while allowing diffusion of one or more reagents used for amplification or analysis (e.g, dNTPs, ddNTPs, enzyme cofactors (e.g., Mg²⁺, Mn²⁺), buffers, ions). The skilled artisan will appreciate that one or more reagents used for amplification or analysis may not be suitable for diffusion within a matrix (e.g. primers, probes, enzymes (e.g., thermostable polymerase)). Therefore, such reagents can be added to the matrix-forming material before the matrix forms. Therefore, in some embodiments, polynucleotides suitable for clonal amplification and analysis can be diluted, as needed, and combined with a matrix-forming material and one or more reagents required for amplification or analysis. A matrix comprising isolated polynucleotides can be allowed to form on a solid support (e.g., a glass slide). However, the skilled artisan will appreciate that other types of supports having various shapes and dimensions also can be utilized. Once isolated with a matrix, a polynucleotide can be amplified by various methods as described below to produce isolated populations of clonal amplicons within the matrix. In some embodiments, polynucleotides isolated within a matrix can be amplified by placing the matrix in a solution comprising an appropriate buffer, pH, and other components that can diffuse into the matrix (e.g., dNTPs, enzyme co-factors, ions (Na⁺, Cl⁻, Mg²⁺)) to provide or maintain suitable amplification conditions.

In some embodiments, polynucleotides suitable for clonal amplification can be isolated by hybridization to a probe attached to a solid support. For example, a solid support (e.g., a glass slide) can comprise a plurality of regions, wherein each region comprises probes suitable for specifically hybridizing to a polynucleotide of interest. Therefore, a polynucleotide of interest can be isolated within each region by contacting the support with a sample under conditions suitable for specific hybridization. In some embodiments, a probe hybridized to a polynucleotide of interest also can function as a primer and, therefore, can be extended by a polymerase to produce a sequence complementary to a polynucleotide of interest. In some embodiments, the polynucleotide of interest can be disassociated from the extended probe and the process can be repeated. As a result, single-stranded clonal amplicons attached to a solid support can be produced. In some embodiments, a primer can be hybridized to a single-stranded clonal amplicon to produce additional copies of the polynucleotide of interest. These additional copies can be hybridized to other probes and provide a template for probe extension. The skilled artisan will appreciate that in some embodiments if a suitable amount of primer is present each of the single-stranded clonal amplicons can be made to be double stranded.

In some embodiments, polynucleotides suitable for clonal amplification can be isolated using recombinant DNA techniques that are well-known in the art. (Sambrook et al., Molecular Cloning: A Laboratory Manual (3d. ed. Cold Spring Harbor Laboratory Press)) For example, in some embodiments, individual polynucleotides can be inserted into recombinant vectors which can be introduced into host cells capable of replicating the vector. The reaction conditions under which polynucleotides are introduced into recombinant vectors can be designed to favor the insertion of a single polynucleotide into each vector. In some embodiments, this can be accomplished by utilizing a concentration of vector that is in molar excess (e.g., ≧10X) of the polynucleotides. Similarly, to favor the transformation of a single host cell by a single vector, host cells can be utilized at a concentration in molar excess of the vectors. In some embodiments, a recombinant vector can be designed to favor the insertion of polynucleotides of interest over other polynucleotides that may be present in a sample. As the skilled artisan will appreciate this can be accomplished by various methods known in the art. For example, in some embodiments, a vector can comprise 5′-single stranded sequences or “sticky-ends” to favor the insertion of polynucleotides having 5′-sequences complementary to the sticky-ends of the vector. As the skilled artisan also will appreciate, various types of selection techniques (e.g., antibiotic susceptibility, metabolic properties (e.g., lactose utilization)) can be utilized to identify and isolate host cells comprising vectors having an inserted polynucleotide. Determining the type of vectors suitable for use with various prokaryotic and eukaryotic host cells is within the abilities of the skilled artisan. Once the recombinant vector comprising an inserted polynucleotide is introduced into an appropriate host cell, the vector and the inserted polynucleotide are clonally amplified by the host cell and then harvested for analysis.

In some embodiments, polynucleotide sequences suitable for clonal amplification can be isolated within a hydrophilic compartment of an inverse emulsion. (U.S. Pat. Nos. 5,616,478, 5,958,698, 6,001,568, 6,432,360, 6,485,944, 6,511,803, 6,440,706, 6,753,147, 6,753,147, U.S. Application Serial Nos. 2002090629, 2002120126, 2002120127, 2002127552, 2003124594; WO0109386; WO0109386; Dressman et al., 2003, Proc. Natl. Acad. Sci. USA 100(15):8817-22; Mitra et al., 1999, Nucleic Acids Res. 27(24):e34; and Shendure et al., 2004, Nat. Rev. Genet. 5(5):335-44, incorporated by reference). “Inverse emulsion”, “water-in-oil emulsion” (“W/O”) and grammatical equivalents as used herein refer to a colloidal composition comprising a discontinuous hydrophilic phase distributed as discrete compartments in a continuous, hydrophobic phase. In various exemplary embodiments, the hydrophilic phase compartments can comprise a semi-solid or matrix material (e.g., agarose, acrylamide) or can comprise an aqueous solution (“aqueous droplet”). As the skilled artisan will appreciate, the dimensions of the hydrophilic compartments in general are not uniform and their average dimensions can be dependent upon several factors, including but not limited to the composition of the hydrophobic and hydrophilic phases and the method used to prepare the emulsion. In various exemplary embodiments, the mean diameter of hydrophilic compartments can be about 0.5 μm, 1 μm, 2 μm, 3 μm, 4 μm, 5 μm, 6 μm, 7 μm, 8 μm, 9 μm, 10 μm, 20 μm, 30 μm, 40 μm, 50 μm, 60 μm, 70 μm, 80 μm, 90 μm, 100 μm, 150 μm, 200 μm, 250 μm, 300 μm, 350 μm, 400 μm, 450 μm to about 500 μm. In various exemplary embodiments, the mean volume of hydrophlic compartments can be about 0.5 μm³ to about 4,000,000 μm³, from about 500 μm³ to about 500,000 μm³, from about 8,000 μm³ to about 200,000 μm³. However, larger and smaller compartments also can be contemplated. Non-limiting examples of factors that can be considered in determining a suitable volume or diameter of a hydrophilic compartment include but are not limited to the clonal amplification conditions, the method of analyzing the clonal amplicons, and the “spot size” of the hydrophilic compartment, as described below.

The composition of the continuous and discontinuous phases of an inverse emulsion can be selected at the discretion of the practitioner. In various exemplary embodiments a continuous phase can be can include an oil (e.g., mineral oil, light mineral oil, silicon oil) or a hydrocarbon (e.g., hexane, heptane, octane, nonane, decane, etc.) and the like. The composition of the various phases can be selected to provide a suitable emulsion under the conditions of clonal amplification. Therefore, “suitable emulsion” and equivalents refer to an emulsion that does not substantially degrade, collapse or in which the hydrophilic compartments do not substantially coalesce under the clonal amplification conditions. Therefore, in various exemplary embodiments, an emulsion can be suitable for carrying out reactions at varying temperatures (e.g., thermocycling, such as, PCR), and other conditions (e.g., pH, ionic strength, hybridization conditions, etc.), and in the presence of various reaction components (e.g., nucleic acids, proteins, enzymes, catalysts, co-factors, intermediates, products, by-products, labels, microparticles, etc.).

In some embodiments an inverse emulsion can comprise compositions or compounds that modify the inverse emulsion's stability. In some embodiments, such compounds can be amphipathic and therefore comprise hydrophobic and hydrophilic groups. In various exemplary embodiments, the hydrophilic group can be polar, positively charged or negatively charged. The skilled artisan can appreciate that amphipathic compounds, depending on their concentration and the composition of the various phases, can be used to increase or decrease the stability of an inverse emulsion. Examples of amphipathic compounds include but are not limited to proteins, polypeptides, and surfactants, such as, detergents and emulsifiers, all of which can be used alone or in any combination. For example, an amphipathic compound can be a protein or polypeptide (e.g., albumin), lecithin, sodium oleate, glycolic acid ethoxylate oleyl ether, 4-(1-aminoethyl)phenol propoxylate, glycolic acid ethoxylate 4-tert-butylphenyl ether, glycolic acid ethoxylate oleyl ether, sodium dodecyl sulfate, 3-[(3-cholamidopropyl)dimethylammonia]-1-propanesulfonate, n-dodecyl-β-D-maltoside (lauryl-β-D-maltoside), n-octyl-β-D-glucopyranoside, n-octyl-β-D-thioglucopyranoside (OTG), 4-(1,1,3,3-tetramethylbutyl)phenol polymer, N-lauroylsarcosine, polyethylene-block-poly(ethylene glycol), sodium 7-ethyl-2-methyl-4-undecyl sulfate, glycolic acid ethoxylate lauryl ether, Altox® 4912, Tween® 20, Tween® 80, sorbitan monooleate (Span 80), Triton® X-100, Triton® X-114, Brij®-35, Brij®-58, 3-[(3-cholamidopropyl)-dimethylammonio]-1-propane-sulfonate (CHAPS), Nonidet P-40 (NP-40). For further description of these and/or other amphipathic compounds and methods of use in emulsions see, e.g., Becher, Emulsions: Theory and Practice, 3rd ed. Oxford University Press 2001 (ISBN 0841234965); Becher (ed.) Encyclopedia of Emulsion Technology: Basic Theory Vol. I-IV, Marcel Dekker Inc. 1983 (ISBN: 0824718763), 1985 (ISBN: 0824718771), 1987 (ISBN: 082471878X), 1996 (ISBN: 0824793803); Holmberg, Surfactants and Polymers in Aqueous Solutions 2nd ed., John Wiley & Sons 2002 (ISBN: 0471498831); Lissant (ed.), Emulsions and Emulsion Technology. Marcel Dekker Inc. 1984 (ISBN: 0824770838); Lissant, Emulsions and Emulsion Technology (Surfactant Science). Marcel Dekker Inc. 1974 (ISBN: 0824760972); Lissant (ed.), Emulsions and Emulsion Technology/Part II (Surfactant Science, Vol. 6). Marcel Dekker Inc. 1974 (ISBN 0824718925); Lissant, Emulsions and Emulsion Technology Marcel Dekker Inc. 1984 (ISBN: 0824790472); Handbook of Industrial Surfactants (ISBN 1890595209).

Methods of making inverse emulsions are known in the art and include but are not limited drop wise addtion of an aqueous solution into a stirred hydrophobic solution optionally comprising one or more amphipathic compounds (see, e.g., Becher, Emulsions: Theory and Practice, 3rd ed. Oxford University Press 2001 (ISBN 0841234965); Becher (ed.) Encyclopedia of Emulsion Technology: Basic Theory Vol. I-IV, Marcel Dekker Inc. 1983 (ISBN: 0824718763), 1985 (ISBN: 0824718771), 1987 (ISBN: 082471878X), 1996 (ISBN: 0824793803); Dressman et al., 2003, Proc. Natl. Acad. Sci. USA. 100(15):8817-22 (Epub 2003 Jul. 11); Ghadessey et al., 2001, Proc. Natl. Acad. Sci. USA. 98:4552-7; Griffiths et al., 2003, EMBO 22:24-35; Lissant (ed.), Emulsions and Emulsion Technology. Marcel Dekker Inc. 1984 (ISBN: 0824770838); Lissant, Emulsions and Emulsion Technology (Surfactant Science). Marcel Dekker Inc. 1974 (ISBN: 0824760972); Lissant (ed.), Emulsions and Emulsion Technology/Part II (Surfactant Science, Vol. 6). Marcel Dekker Inc. 1974 (ISBN 0824718925); Lissant, Emulsions and Emulsion Technology Marcel Dekker Inc. 1984 (ISBN: 0824790472); Nakano et al., 2003, J. Biotechnol. 102(2):117-24; Tawfik et al., 1998, Nat. Biotechnol. 16(7):652-6; U.S. Pat. No. 6,489,103; and WO 2002/22869). Therefore, in some embodiments, polynucleotides and reagents suitable for clonal amplification or analysis can be isolated within hydrophilic compartments by drop-wise addition of an aqueous solution comprising the polynucleotides and such reagents into a stirred hydrophobic solution. In some embodiments, the polynucleotide concentration of the aqueous solution can be adjusted such that hydrophilic compartments of the inverse emulsion average from >0 to <1 polynucleotide per compartment.

In some embodiments, emulsion formation can be monitored by high-resolution ultrasonic spectroscopy in which changes in ultrasonic velocity and attenuation that occur as a function of time are indicative of emulsion formation, as known in the art. In some embodiments, the size (e.g., mean droplet diameter), number, and/or composition of the hydrophilic compartments can be analyzed to sort or remove hydrophilic compartments unsuitable for clonal amplification or analysis. Therefore, in some embodiments, probes (e.g., molecular beacons), primers (e.g., scorpions), labels (fluorescent molecules) and other moieties (e.g., magnetic beads) can be included in the hydrophilic compartments to provide a detectable signal or moiety that can be used to identify hydrophilic compartments of interest. Therefore, methods suitable for sorting hydrophilic compartments include but are not limited to microscopic examination (Vogelstein et al., 1999, Proc. Natl. Acad. Sci. USA 96:9236-9241; Dressman et al., 2003, Proc. Natl. Acad. Sci. USA. 100(15):8817-22 (Epub 2003 Jul. 11) or laser diffraction (Tawfik et al., 1998, Nat. Biotechnol. 16(7):652-6), laser Doppler velocimetry/anemometry (“LDV” or “LDA”), flow cytometry, microflow cytometry, affinity chromatography (e.g., columns and/or pads), exposure to magnetic fields, and the like.

In some embodiments, the aqueous solution comprising the polynucleotides that can be used to form an inverse emulsion also can comprise all of the reagents suitable for clonal amplification. Therefore, in some embodiments, when the inverse emulsion is formed it can be incubated under conditions suitable to clonally amplify the polynucleotides isolated within the various compartments. In some embodiments, one or more clonal amplification reagents can be omitted from the aqueous solution comprising the polynucleotides used to form the inverse emulsion. Therefore, in some embodiments, these and other reagents, such as reagents suitable for detection or analysis of the clonal amplicons, can be introduced into the hydrophilic compartments after the inverse emulsion is formed as described below.

Once the polynucleotides are isolated they can be clonally amplified using the principals and techniques of various methods known in the art. Selecting a method suitable for clonal amplification of isolated polynucleotides is within the abilities of the skilled artisan. Methods suitable for clonal amplification include but are not limited to PCR (see, e.g., U.S. Pat. Nos. 4,683,195, 4,683,202, 4,800,159, 4,965,188, 5,075,216, 5,176,995, 5,338,671, 5,386,022, 5,333,675, 5,340,728, 5,405,774, 5,436,149, 5,512,462, 5,618,703, 5,656,493, 6,037,129, 6,040,166, 6,197,563, 6,300,073, 6,406,891, 6,514,736; EP-A-0200362, EP-A-0201184, U.S. Application Ser. No. 60/584,665, Edwards et al. (eds.), 2004, Real-Time PCR: An Essential Guide. Horizon Bioscience Norfolk, UK (ISBN 0-9545232-7-X)), LCR (see, e.g., EP-A-320308 and U.S. Pat. Nos. 5,427,930, 5,516,663, 5686272, and 5869252), OLA (see, e.g. U.S. Pat. Nos. 4,883,750, 5,242,794, 5521065, 5962223; Brinson et al., 1997, Genet. Test. 1(1):61-8. Erratum in: Iovannisci, 1998, Genet. Test. 2(4):385; Grossman et al., 1994, Nucleic Acids Res. 22(21):4527-34. Erratum in: Iovannisci, 1998, Nucleic Acids Res. 26(23):5539; lannone et al., 2000, Cytometry 39(2):131-40; Nickerson et al., 1990, Proc. Natl. Acad. Sci. USA. 87(22):8923-7), Q-beta amplification (see, e.g. U.S. Pat. Nos. 4,786,600, 4,9578, 585,356,774, 5,364,760, 5,503,979, 5,602,001, 5620851; “Amplifying Probe Assays with Q-Beta Replicase” Bio/Technology 1989: 7(6), 609-10 (Eng.); Pritchard, 1990, J. Clin. Lab. Anal. 4:318), NASBA™ (Burchill et al., 2002, Br. J. Cancer. 86(1):102-9; Deiman et al., 2002, Mol. Biotechnol. 20(2):163-79; Malek et al. “Nucleic Acid Sequence-Based Amplification (NASBA™)” Ch. 36 In Methods in Molecular Biology, Vol. 28: Protocols for Nucleic Acid Analysis by Nonradioactive Probes, Isaac (ed.) Humana Press, Inc., Totowa, N.J. (1994); Romano et al., 1997, Immunol. Invest. 26(1-2):15-28), strand displacement amplification ((SDA) U.S. Pat. Nos. 5,270,184 and 5,455,166; Walker. “Empirical Aspects of Strand Displacement Amplification” In PCR Methods and Applications, 3(1):1-6 Cold Spring Harbor Laboratory Press, Cold Spring Harbor, N.Y. (1993), rolling circle amplification (RCA), transcription, and reverse transcription.

In some embodiments, isolated polynucleotides can be clonally amplified using a primer attached to a solid support or surface (e.g., a chip, slide, membrane, gel). Therefore, in some embodiments, clonal amplification reactions can yield a surface comprising a plurality of isolated clonal amplicons. In various exemplary embodiments, a solid support may have a wide variety of forms, including membranes, slides, plates, micromachined chips, microparticles, beads and the like. Solid supports may comprise a wide variety of compositions including, but not limited to, glass, plastic, silicon, alkanethiolate derivatized gold, cellulose, low cross linked and high cross linked polystyrene, silica gel, polyamide, and the like, and can have various shapes and features (e.g., wells, indentations, channels, etc.). Methods of attaching primers to a surface are known in the art (see, e.g., Beier et al., 1999, Nucleic Acids Res. 27(9):1970-1977; Brison et al., 1982, Molecular and Cellular Biology 2:578 587; Cheung et al., 1999, Nat. Genet 21(1 Suppl):15-19; Chrisey et al., 1996, Nucleic Acids Res. 24(15):3031-3039; Cohen et. al., 1997, Nucleic Acids Res. 1997 Feb. 15; 25(4):911-912; Devivar et al., 1999, Bioorg. Med. Chem. Lett. 9(9):1239-1242; Heme et al., 1997. J. Am. Chem. Soc. 119:8916-8920; Kumar et al., 2000, Nucleic Acis Res. 28(14):e71; Lipshutz et al., 1999, Nat. Genet. 21(1 Suppl):20-24; Milner et al., 1997, Nat. Biotechnol. June;15(6):537-541; Morozov et al., 1999, Anal. Chem. 71(15):3110-3117; Proudnikov et al., 1998, Anal Biochem. 259(1):34-41; Rasmussen et al., 1991, Anal Biochem. 198(1):138-142; Rogers et al., 1999, Anal. Biochem. 266(1):23-30; Salo et al., 1999, Bioconjug Chem. 10(5):815-823; Singh-Gasson et al., 1999, Nat. Biotechnol. 17(10):974-978, and Pierce Chemical Company Catalog 1994, pp. 155-200), incorporated herein by reference).

A non-limiting example of the use of a primer attached to a microparticle to clonally amplify a polynucleotide in a hydrophilic compartment of an inverse emulsion is illustrated in FIG. 2. In FIG. 2, are shown inverse emulsion 120 comprising hydrophobic phase 125 and a plurality of hydrophilic compartments 130A-D. Hydrophilic compartment 130A contains isolated polynucleotide 200, multiple copies of reverse primer 80, and multiple copies of forward primer 100 attached to microparticle 90. Therefore, FIG. 2 illustrates an embodiment wherein the entire surface to which a primer is attached is completely contained, confined, or encased within a hydrophilic compartment. Clonal amplification of polynucleotide 200 by PCR will yield an isolated population of double stranded clonal amplicons 105 attached to microparticle 90.

To analyze double stranded clonal amplicons attached to microparticles, the emulsion can be collapsed and the microparticles can be collected. Methods of collapsing an inverse emulsion are known in the art and include but are not limited to modifying the concentration of an amphipathic compound in the emulsion and centrifugation. In some embodiments, the double stranded clonal amplicons can be denatured, leaving one strand of the clonal amplicons attached to the microparticles. In some embodiments, the microparticles can be distributed into wells of a multi-well plate and analyzed as disclosed herein.

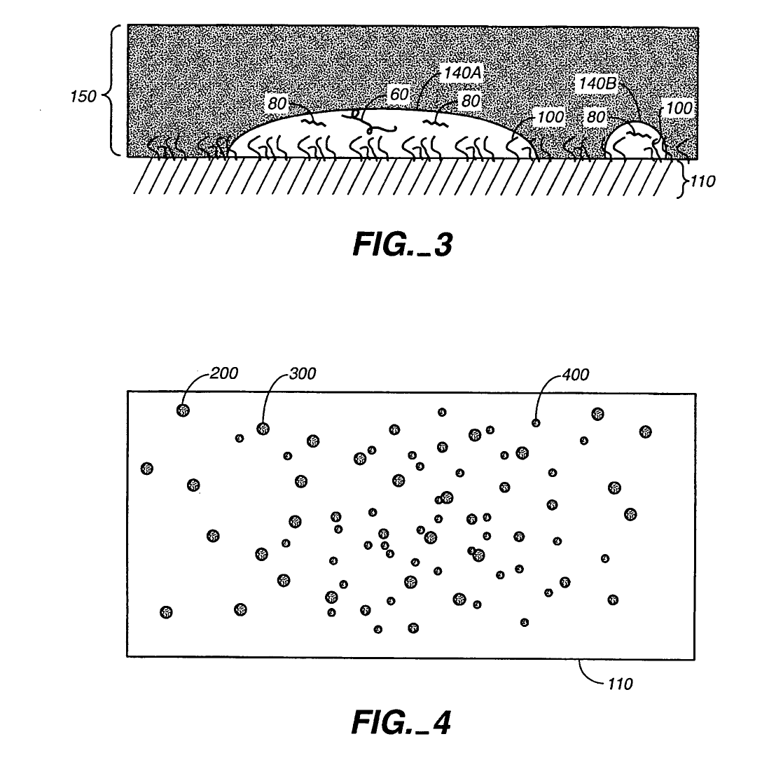

A non-limiting example of the use of primers attached to a surface to clonally amplify a polynucleotide in a hydrophilic compartment of an inverse emulsion is illustrated in FIG. 3. In FIG. 3, are shown inverse emulsion 150 comprising a plurality of hydrophilic compartments 140A-B disposed upon surface 110. Compartment 140A contains isolated target polynucleotide 60, multiple copies of reverse primer 80, and multiple copies of forward primer 100 attached to surface 110. Therefore, FIG. 3 illustrates an embodiment wherein a hydrophilic compartment is disposed upon the surface to which a primer is attached. Although, FIG. 3 illustrates an embodiment in which a primer can be attached to a surface, other reagents suitable for amplification and analysis also can be attached to the surface at the discretion of the practitioner. In some embodiments, a surface can contain a coating of film of lyophilized or partially lyophilized reagents which can become reconstituted when contacted with a hydrophilic compartment. Clonal amplification of polynucleotide 60 will yield an isolated population of double stranded clonal amplicons attached to surface 100 that are restricted to the area corresponding to the “spot-size” of hydrophilic compartment 140A. This is further shown in FIG. 4, which illustrates an embodiment in which a plurality of isolated populations of clonal amplicons 200, 300, 400 can be attached to discrete locations of surface 110.

In some embodiments, a primer can be attached to a surface and can project into an aqueous compartment without the aqueous compartment contacting the surface. Therefore, in some embodiments the surface can be external to the aqueous compartment. In some embodiments, electrostatic forces from for example an attachment moiety or linker, and/or the surface can prevent an aqueous compartment from contacting the surface.

In some embodiments, polynucleotides that can be isolated, clonally amplified, and analyzed by the disclosed methods can be specific regions of a complex polynucleotide (e.g., a chromosome) or selected from complex mixtures of polynucleotides (e.g., a genome; nucleic acid libraries, etc.). Thus, various aspects or characteristics of complex polynucleotides, such as a genome or a cell or organism, can be specifically targeted and analyzed. Non-limiting examples of genomic regions that can be specifically targeted include but are not limited to cis-acting regulatory elements, regions of rearrangement (e.g., antibody and T-cell receptor genes, oncogenes), recombination, insertion (e.g. viral insertion, e.g., retroviral insertion), deletion, gene duplication, transpositional elements, highly-repetitive sequences, specific genes (e.g., genes that encode RNA or protein (e.g., cell cycle regulators, transcription factors, replication/repair proteins, etc.), pseudogenes, transcribed genes (e.g., the transcriptome), genomic regions associated with a disease state (e.g., cancer, cognitive disorders, birth defects, drug addiction, psychiatric disorders, autoimmune disease etc.) can be selectively analyzed by the disclosed methods. In some embodiments, specific regions of a genome can be selected and analyzed at any one or more stages of a cell cycle, or differentiation, or in response to natural (e.g., antigens, cytokines, hormones, etc.) and/or artificial stimuli (e.g., carcinogens, mutagens, pharmaceuticals etc.). Thus, in some embodiments, the methods disclosed herein can be used to selectively determine the expression and/or transcription profile of one or more cells by selectively targeting genomic regions of interest. In various exemplary embodiments, about 1, 5, 10, 15, 20, 25, 30, 35, 40, 45, 50, 55, 60, 65, 70, 76, 80, 85, 90, 95 or about 100% of a genome can be analyzed by the disclosed methods.

In some embodiments, polynucleotides can be selected for clonal amplification by multiplex amplification of polynucleotide sequences. Therefore, in some embodiments, the disclosed methods can comprise multiplex amplification of polynucleotide sequences to produce a heterogeneous mixture of amplicons (“non-clonal amplicons” or “multiplex amplicons”). Once made, the multiplex amplicons can be isolated, clonally amplified, and analyzed.

In various exemplary embodiments, multiplex amplicons can be made by PCR amplification, which can include exponential, linear, asymmetric, and/or log-linear PCR (see, e.g., U.S. Application Ser. No. 60/584,665). In some embodiments, multiplex PCR amplification conditions can be designed to reach a plateau. “Plateau” herein refers to the stage of an amplification reaction (e.g., PCR) when synthesis and consequent accumulation of amplicons may terminate even though primers, template, polymerase and dNTPs can be present. This can occur when hybridization of the first and second strands of double-stranded amplicons to each other competes with the hybridization of the amplification primers to the individual amplicon strands. In some embodiments, a plateau can occur when one or more reagents are consumed (see, e.g., Saunders, Quantitative Real-Time PCR 106, 108 (Edwards et al. eds., 2004 (Horizon Bioscience, Norfolk UK, ISBN 0-9545232-7-X)); and Bustin et al., Analysis of mRNA Expression by Real-Time PCR 127 (Edwards et al. eds., 2004 (Horizon Bioscience, Norfolk UK, ISBN 0-9545232-7-X))). However, in some embodiments, amplification conditions can be designed to terminate before a reaction would otherwise reach a plateau. In some embodiments, terminating amplification before reaching a plateau can minimize amplification of polynucleotides that are most abundant in a sample. Therefore, in some embodiments, an equivalent number of multiplex amplicons from various polynucleotide can be produced irrespective of the starting copy number of the various polynucleotides. In some embodiments, terminating a PCR reaction before a plateau can be achieved using a limiting and equivalent number of amplification primer pairs for each target polynucleotide to be analyzed (see, e.g., U.S. Application Ser. No. 60/584,665).

In some embodiments, multiplex amplicons can be produced by PCR as described in U.S. Patent Application No. 20040175733, incorporated by reference. Therefore, in some embodiments, the conditions of multiplex amplification can include a concentration of thermostable polymerase, such as, AMPLITAQ GOLD™ DNA polymerase (Applied Biosystems, Applera Corp., Foster City, Calif.) from about 2 U/20 μl to about 16 U/20 μl, from about 2 U/20 μl to about 9 U/20 μl, from about 2 U/20 μl to about 6 U/20 μl, from about 7 U/20 μl to about 16 U/20 μl, or from about 2, 3, 4, 5, 6, 7, 8, 9, 10, 11, 12, 13, 14, 15, or 16 U/20 μl reaction volume. In some embodiments, primer extension can be for about 2, 3, 4, 5, 6, 7, 8, 9, 10 min., or even longer. In some embodiments, multiplex amplification primers can be used at concentrations in the range of about 30-900 nM each primer. Different amplification primer pairs may be present at different concentrations within this range or, alternatively, some or all of the multiplex amplification primers may be present at approximately equimolar concentrations within this range. In some embodiments, at least some of the multiplex amplification primers, for example, approximately 10%, 25%, 35%, 50%, 60%, or more, can be present in approximately equimolar concentrations ranging from about 30 nM to about 100 nM each primer. In some embodiments, all of the multiplex amplification primers can be present at approximately equimolar concentrations in the range of about 30 nM to about 100 nM each primer. In some embodiments, all of the amplification primers can be present at concentrations of about 10, 20, 30, 40, 45, 50, 60, 70, 80, 90, 100, 200, 300, 400, 500, 600, 700, 800 or 900 nM each primer. In some embodiments, some or all of the amplification primers can be present in a concentration of about 45 μM each primer. The amplification primer concentrations discussed above can be used regardless of whether the target polynucleotide(s) being amplified are RNA or DNA. In some embodiments, the number of primer pairs can be at least 100, 200, 300, 400, 500, 600, 700, 800, 900, 1000, 2000, 3000, 4000, 5000, 6000, 7000, 8000, 9000, 10000, 15000, 20000, 25000 or up to about 30000. In addition, in embodiments wherein targeted polynucleotides are RNA, the reverse-transcription reaction of a multiplex RT-PCR amplification works well at the stated primer concentrations.

The number of multiplex amplification cycles performed may depend upon, among other factors, the degree of amplification desired, which may depend upon such factors as the amount of polynucleotide to be multiplex amplified and/or the intended method of clonal amplification and analysis. Accordingly, the number of cycles employed can vary for different applications and will be apparent to those of skill in the art. For most applications, multiplex amplification reactions carried out for about 10 amplification cycles can be expected to yield sufficient amplification product even when the sample is of limited quantity (e.g., 1 to a few cells), a polynucleotide of interest is present in very low copy number, and/or is present only as a single copy, regardless of the amount of sample required to perform the analysis. However, more or fewer multiplex amplification cycles may be employed. In some embodiments multiplex amplification can be carried out for as many as 1, 2, 3, 4, 5, 6, 7, 8, 9, 10, 11, 12, 13, 14, 15, 16, 17, 18, 19, 20, or more cycles. In some embodiments, multiplex amplification can be carried out for 2-12 cycles, inclusive, for 5-11 cycles, inclusive, or for up to 14 cycles, inclusive.

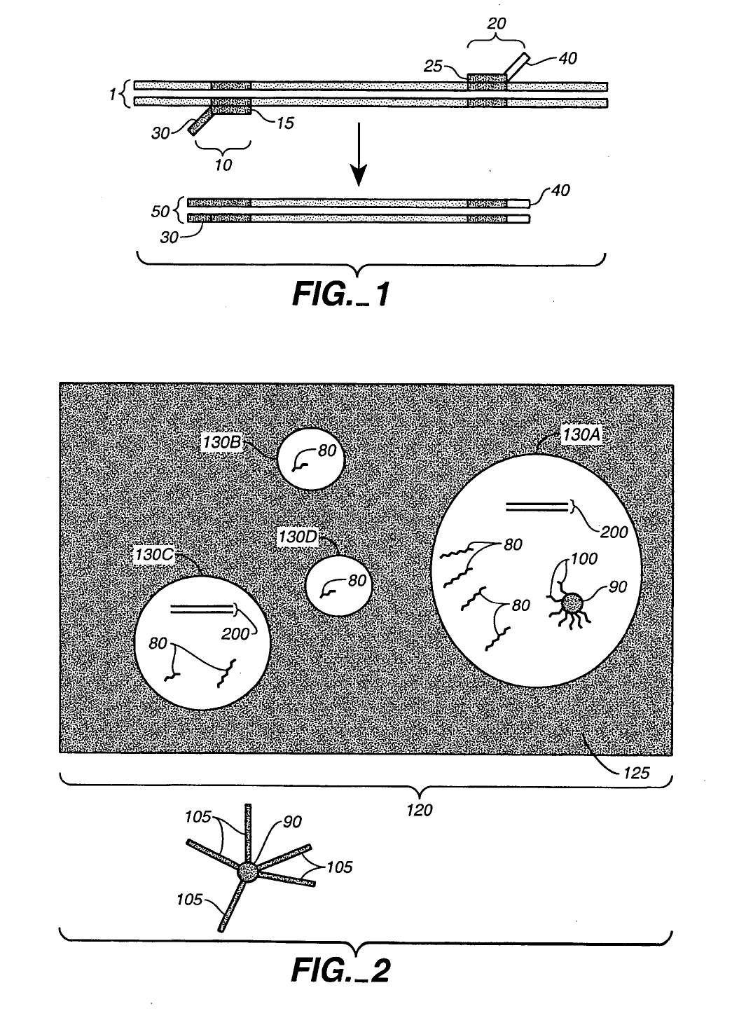

In addition to sequences suitable for priming multiplex amplification of polynucleotides, one or more multiplex amplification primers can be designed to introduce sequences into multiplex amplicons that can be used to facilitate isolation, clonal amplification, and analysis. However, the skilled artisan will appreciated that other types of sequences also can be introduced into multiplex amplicons, such as, enhancers, promoters, restriction endonuclease sites, etc. In some embodiments, a sequence introduced into a multiplex amplicon may be a code sequence which may be used as a surrogate or marker for each multiplex amplicon. Therefore, each “code sequence” is substantially unique and can be used to identify or distinguish the polynucleotide comprising the code sequence (see, e.g., U.S. Application Ser. Nos. 60/584,596; 60/584,621; 60/584,643; 60/584,665). In some embodiments, a multiplex amplification primer sequence may be shared by at least one other amplicon. For example, in some embodiments, a sequence may be common to each forward amplification primer or each reverse amplification primer. Thus, “forward universal sequence” and “reverse universal sequence” refer to multiplex amplification primer sequences shared by each forward or reverse primer, respectively. As exemplified in FIG. 1, forward primer 10 and reverse primer 20 comprise polynucleotide 1 specific sequences 15 and 25, respectively. 5′ relative to polynucleotide 1 specific sequences, forward primer 10 and reverse primer 20 comprise universal forward sequence 30 and universal reverse sequence 40, respectively, which are incorporated into amplicon 50. Therefore, when amplicon 50 is isolated, for example, in a hydrophilic compartment, the universal forward and reverse can be used as primer sites for clonal amplification or analysis.

In some embodiments, code, universal, and/or other types of sequences can be added to a polynucleotide or multiplex amplicons using linkers and/or adaptors (Sambrook et al., Molecular Cloning: A Laboratory Manual 1.84, 1.88-1.89, 1.98-1.102, 1.160-1.161, 11.20-11.21, 11.51-11.55, 11.102 (3d. ed. Cold Spring Harbor Laboratory Press). For example, in some embodiments, a polynucleotide can be sheared, restriction enzyme digested, or treated with a polymerase or kinase to prepare the termini of a polynucleotide for the addition of linkers and/or adaptors. In some embodiments, sequences, including those described above, can be added to a polynucleotide by homologous recombination using RecA and/or other recombinases (see, e.g., U.S. Pat. Nos. 4,888,274, 5,989,879, 6,090,539, 6,074,853, 6,200,812, 6,391,564, 6,524,856). Determining the number, type, length, and composition of the various sequences and their distribution or commonality among polynucleotides or multiplex amplicons employed, including incorporation of sequences into amplification primers and amplicons derived therefrom are known in the art. (see, e.g., U.S. Pat. Nos. 5,314,809, 5,853,989, 5,882,856, 6,090,552, 6,355,431, 6,617,138, 6,630,329, 6,635,419, 6,670,130, 6,670,161; and Weighardt et al., 1993, PCR Methods and App. 3:77, the disclosures of which are incorporated by reference).

As will be appreciated by skilled artisans, @polynucleotides suitable for analysis by the disclosed methods may be either DNA (e.g, cDNA, genomic DNA, extrachromosomal DNA (e.g. mitochondrial DNA, plasmid DNA), an amplicon) or RNA (e.g., mRNA, rRNA, tRNA, an in vitro transcript, or genomic RNA (e.g., virion RNA (vRNA)) in nature, and may be derived or obtained from virtually any sample or source (e.g., human, non-human, plant, animal, microorganism etc.), wherein the sample may optionally be scarce or of a limited quantity. For example, the sample may be one or a few cells collected from a crime scene or a small amount of tissue collected via biopsy. In some embodiments, the target polynucleotide may be a synthetic polynucleotide comprising nucleotide analogs or mimics, as described below, produced for purposes, such as, diagnosis, testing, or treatment.

In various non-limiting examples, polynucleotide suitable for analysis may be single or double-stranded, or a combination thereof, linear or circular, a chromosome or a gene or a portion or fragment thereof, a regulatory polynucleotide, a restriction fragment from, for example, a plasmid or chromosomal DNA, genomic DNA, mitochondrial DNA, DNA from a construct or library of constructs (e.g., from a YAC, BAC or PAC library), RNA (e.g, mRNA, rRNA or vRNA) or a cDNA or a cDNA library. As known in the art, a cDNA is a single- or double-stranded DNA produced by reverse transcription of an RNA template. Therefore, some embodiments include a reverse transcriptase and one or more primers suitable for reverse transcribing an RNA template into a cDNA. Reactions, reagents and conditions for carrying out such “RT” reactions are known in the art (see, e.g., Blain et al., 1993, J. Biol. Chem. 5:23585-23592; Blain et al., 1995, J. Virol. 69:4440-4452; PCR Essential Techniques 61-63, 80-81, (Burke, ed., J. Wiley & Sons 1996); Gubler et al., 1983, Gene 25:263-269; Gubler, 1987, Methods Enzymol., 152:330-335; Sellner et al., 1994, J. Virol. Method. 49:47-58; Okayama et al., 1982, Mol. Cell. Biol. 2:161-170; and U.S. Pat. Nos. 5,310,652, 5,322,770, and 6,300,073, these disclosures of which are incorporated herein by reference. In some embodiments, a polynucleotide may include a single polynucleotide (e.g., a chromosome, plasmid) from which one or more different sequences of interest may be optionally selected, clonally amplified, and analyzed.

In some embodiments, clonal amplicons can be analyzed by virtually any method selected at the discretion of the practitioner. Therefore, reactions comprising any one or more steps of probe or primer hybridization, primer extension, labeling, etc. can be used to detect, quantitate, and/or determine the composition of clonal amplicons. For example, in some embodiments, the transcriptome of one or more genomes can be amplified by multiplex PCR, as described above, whereby forward and reverse universal amplicons can be incorporated into each amplicon. In some embodiments, the multiplex amplicons can be isolated, for example, in hydrophilic compartments of an inverse emulsion, and clonally amplified using primers comprising the forward and reverse universal sequences. In some embodiments, one of the clonal amplification primers can be attached to a surface as exemplified in FIG. 3 to produce isolated populations of clonal amplicons as exemplified in FIG. 4.

In some embodiments, clonal amplicons can be analyzed in a parallel manner. Without being bound by theory, because the clonal amplicons that are produced are isolated as discrete populations, the clonal amplicons can be analyzed in parallel. For example, as shown in FIG. 4, the discrete populations of clonal amplicons can be analyzed in parallel as a result of their attachment to discrete areas of a surface. Therefore, in some embodiments, at least at least 100, 500, 1000, 10000, 50000, 100000, 300000, 500000, or 1000000 populations of clonal amplicons can be analyzed in parallel. The skilled artisan will appreciate that various methods can be suitable for parallel analysis of clonal amplicons. Generally, such methods can produce a discrete detectable signal that can be associated or linked to individual populations of clonal amplicons.

In some embodiments, clonal amplicons can be sequenced using sequencing techniques based on sequencing-by-synthesis techniques. For example, in some embodiments the enzymatic method of Sanger et al. 1977, Proc. Natl. Acad. Sci., 74: 5463-5467, can be employed. The Sanger technique uses controlled synthesis of nucleic acids to generate fragments that terminate at specific points along the sequence of interest. Techniques based on the Sanger method typically begin by annealing a synthetic sequencing primer to a nucleic acid template (e.g., target polynucleotide or amplicon). The primer can be extended in the presence of four dNTPs (i.e., dGTP, dCTP, dATP and dTTP) and small proportion of four 2′,3′-ddNTPs that carry a 3′-H atom on the deoxyribose moiety, rather than the conventional 3′-OH group. Incorporation of a ddNTP molecule into the growing DNA chain prevents formation of a phosphodiester bond with the succeeding dNTP, thus, extension of the growing chain can be terminated. The products of the reaction are a nested set of oligonucleotide chains with co-terminal 5′ termini and whose lengths are determined by the distance between the 5′ terminus of the primer used to initiate DNA synthesis and the sites of ddNTP chain termination. These populations of oligonucleotides can be separated by electrophoresis and the sequence of the template DNA determined (see, e.g., U.S. Pat. Nos. 4,994,372, 5,332,666, 5,498,523, 5,800,996, 5,821,058, 5,863,727, 5,945,526, and 6,258,568; and Sanger et al., 1972, Proc. Natl. Acad. Sci. USA, 74: 5463-5467; and Sanger, 1981, Science, 214: 1205-1210).

Based on the labeling strategy used to identify the bases, described below, sequencing reactions can be performed in parallel. For example, in some embodiments distinguishable labels can be attached to each ddNTP. Therefore, a single extension/termination reaction can be used which contains the four ddNTPs, each comprising a spectrally resolvable label. Suitable spectrally resolvable labels include but are not limited fluorophores. (see, e.g., U.S. Pat. Nos. 5,821,058, 5,332,666, and 5,945,526.)

In some embodiments, a method of sequencing based on the detection of base incorporation by the release of a pyrophosphate and simultaneous enzymatic nucleotide degradation can be used (see, e.g., U.S. Pat. No. 6,258,568). For example, clonal amplicons can be sequenced using a primer and adding four different dNTPs or ddNTPs subjected to a polymerase reaction. As each dNTP or ddNTP is added to the primer extension product, a pyrophosphate molecule is released. Pyrophosphate release can be detected enzymatically, such as, by the generation of light in a luciferase-luciferin reaction (see, e.g., WO 93/23564 and WO 89/09283). Additionally, a nucleotide degrading enzyme, such as apyrase, can be present during the reaction in order to degrade unincorporated nucleotides (see, e.g., U.S. Pat. No. 6,258,568; hereby incorporated by reference in its entirety). In other embodiments, the reaction can be carried out in the presence of a sequencing primer, polymerase, a nucleotide degrading enzyme, deoxynucleotide triphosphates, and a pyrophosphate detection system comprising ATP sulfurylase and luciferase (see, e.g., U.S. Pat. No. 6,258,568).

In some embodiments, a method of sequencing can be fluorescent in situ sequencing (FISSEQ). In FISSEQ, a primer can be extended by adding a fluorescently-labeled dNTP followed by washing away of unincorporated dNTP. The incorporated dNTP can be detected by fluorescence. At each cycle, the fluorescence from previous cycles can be “bleached” or digitally subtracted. (Mitra et al., 2003, Analytical Biochemistry 320:55-65; Zhu et al., 2003, Science 301:836-8; U.S. Application Nos. 20020120126, 20020120127, 20020127552, 20030099972, 20030124594, and 20030207265). In some embodiments, a method of sequencing can be hybridization sequencing (see, e.g., Baines et al., 1988, J. Theor. Biol. 135(3):303-7; Drmanac et al., Genomics 4(2):114-28; Khrapko et al., 1989, FEBS Lett. 256(1-2):118-22; Lysov et al.,1988, Dokl Akad Nauk SSSR. 303(6):1508-11; Pevzner, 1989, J. Biomol. Struct. Dyn. 7(1):63-73); Southern et al., 1992, Genomics 13(4):1008-17).

In some embodiments, clonal amplicons attached to a solid support can be sequenced. For example, clonal amplicons attached to a microparticle produced in a hydrophilic compartment can be collected en masse by breaking the emulsion, distributed into individual wells of a multi-well plate, and sequenced. In some embodiments, clonal amplicons attached to a surface of a slide can be sequenced in a parallel reaction.

In some embodiments, clonal amplicons can be sequenced by massively parallel signature sequencing (MPSS) which comprises two techniques: one for tagging and sorting fragments of DNA for parallel processing, and another for the stepwise sequencing the end of a DNA fragment. MPSS is described in U.S. Pat. Nos. 5,599,675, 5,695,934, 5,714,330, 5,763,175, 5,831,065. 5,863,722, 6,013,445, 6,172,214, 6,511,802; U.S. Patent Application Nos. 20040038283, 20040002104, 20030077615; and International Appl. Nos. PCT/US96/09513, PCT/US97/09472. In some embodiments, MPSS can be carried out by ligating an encoded adaptor to an end of a polynucleotide to be sequenced, the encoded adaptor having a nuclease recognition site of a nuclease whose cleavage site is separate from its recognition site; identifying one or more nucleotides at the end of the fragment by the identity of the encoded adaptor ligated thereto, cleaving the polynucleotide with a nuclease recognizing the nuclease recognition site of the encoded adaptor such that the polynucleotide is shortened by one or more nucleotides; and repeating the steps until the nucleotide sequence of the end of the polynucleotide can be determined. (U.S. Pat. No. 6,511,802)

A variety of nucleic acid polymerases may be used in the methods described herein. For example, the nucleic acid polymerizing enzyme can be a thermostable polymerase or a thermally degradable polymerase. Suitable thermostable polymerases include, but are not limited to, polymerases isolated from Thermus aquaticus, Thermus thermophilus, Pyrococcus woesei, Pyrococcus furiosus, Thermococcus litoralis, and Thermotoga maritima. Therefore, in some embodiments, “cycle sequencing” can be performed. Suitable thermodegradable polymersases include, but are not limited to, E. coli DNA polymerase I, the Klenow fragment of E. coli DNA polymerase I, T4 DNA polymerase, T5 DNA polymerase, T7 DNA polymerase, and others. Examples of other polymerizing enzymes that can be used in the methods described herein include but are not limited to T7, T3, SP6 RNA polymerases and AMV, M-MLV and HIV reverse transcriptases.

Non-limiting examples of commercially available polymerases that can be used in the methods described herein include, but are not limited to, TaqFS®, AmpliTaq CS (Perkin-Elmer), AmpliTaq FS (Perkin-Elmer), Kentaq1 (AB Peptide, St. Louis, Mo.), Taquenase (Scien Tech Corp., St. Louis, Mo.), ThermoSequenase (Amersham), Bst polymerase, Vent_{R}(exo⁻) DNA polymerase, Reader™Taq DNA polymerase, VENT™ DNA polymerase (New England Biolabs), DEEPVENT™ DNA polymerase (New England Biolabs), PFUTurbo™ DNA polymerase (Stratagene), Tth DNA polymerase, KlenTaq-1 polymerase, SEQUENASE™ 1.0 DNA polymerase (Amersham Biosciences), and SEQUENASE 2.0 DNA polymerase (United States Biochemicals).

The products of sequencing reactions can be analyzed by a wide variety of methods. For example, the products can be separated by a size-dependent process, e.g., gel electrophoresis, capillary electrophoresis (CE: e.g., 3730 DNA Analyzer, 3730x1 DNA Analyzer, 3100-Avant genetic analyser, and 270A-HT Capillary Electrophoresis system (Applied Biosystems, Foster City, Calif.)) (see, e.g., U.S. Pat. Nos. RE37941, 5,384,024, 6,372,106, 6,372,484, 6,387,234, 6,387,236, 6,402,918, 6,402,919, 6,432,651, 6,462,816, 6,475,361, 6,476,118, 6,485,626, 6,531,041, 6,544,396, 6,576,105, 6,592,733, 6,596,140, 6,613,212, 6,635,164, and 6,706,162) using various polymers (e.g., separation polymer (e.g., POP-4™ POP-6™, or POP-7™ (Applied Biosystems, Foster City, Calif.), linear polyacrylamide (LPA: Klepamik et al., 2001, Electrophoresis 22(4):783-8; Kotler et al., 2002, Electrophoresis 23(17):3062-70; Manabe et al., 1998, Electrophoresis 19:2308-2316)), chromatography, thin layer chromatography, or paper chromatography. The separated fragments can be detected, e.g., by laser-induced fluorescence (see, e.g., U.S. Pat. Nos. 5,945,526, 5,863,727, 5,821,058, 5,800,996, 5,332,666, 5,633,129, and 6,395,486), autoradiagraphy, or chemiluminescence. In some embodiments, the products of the sequencing reaction can be separated using gel electrophoresis and visualized using stains such as ethidium bromide or silver stain. The reaction products can also be analyzed by mass spectrometric methods (see, e.g., U.S. Pat. Nos. 6,225,450 and 5,104,12). In some embodiments, products of the sequencing reaction can be analyzed using microfluidic systems, including but not limited to microcapillary electrophoretic systems and methods (see, e.g., Doherty et al., 2004, Analytical Chemistry 76:5249-5256; Ertl et al., 2004, Analytical Chemistry 76:3749-3755; Haab et al., 1999, Analytical Chemistry 71:5137-5145 (1999); Kheterpal et al., 1999, Analytical Chemistry 71:31A-37A; Lagally et al., 2000, Sensors and Actuators B 63:138-146; Lagally et al., 2001, Anal. Chem. 73:565-570; Lagally et al., 2003, Genetic Analysis Using a Portable PCR-CE Microsystem, in Micro Total Analysis Systems Vol. 2, Northrup et al. (eds.) pp. 1283-1286; Liu et al., 1999, Anal. Chem. 71:566-573; Medintz et al., 2000, Electrophoresis 21:2352-2358; Medintz et al., 2001, Genome Research 11:413-421; Paegel et al., Current Opinions in Biotechnology 14:42-50; Scherer et al., 1999, Electrophoresis 20:1508-1517; Shi et al., 1999, Analytical Chemistry 71:5354-5361; Wedemayer et al., 2001, BioTechniques 30:122-128; U.S. Pat. Nos. 6,787,015, 6,787,016; U.S. Application Nos. 20020166768, 20020192719, 20020029968, 20030036080, 20030087300, 20030104466, 20040045827, 20040096960; EP1305615; and WO 02/08744).

The various primers (e.g., multiplex amplification, clonal amplification, and/or sequencing), generally, should be sufficiently long to prime template-directed synthesis under the conditions of the reaction. The exact lengths of the primers may depend on many factors, including but not limited to, the desired hybridization temperature between the primers and polynucleotides, the complexity of the different target polynucleotide sequences, the salt concentration, ionic strength, pH and other buffer conditions, and the sequences of the primers and polynucleotides. The ability to select lengths and sequences of primers suitable for particular applications is within the capabilities of ordinarily skilled artisans (see, e.g., Sambrook et al. Molecular Cloning: A Laboratory Manual 9.50-9.51, 11.46, 11.50 (2d. ed., Cold Spring Harbor Laboratory Press); Sambrook et al., Molecular Cloning: A Laboratory Manual 10.1-10.10 (3d. ed. Cold Spring Harbor Laboratory Press)). In some embodiments, the primers contain from about 15 to about 35 nucleotides that are suitable for hybridizing to a target polynucleotide and form a substrate suitable for DNA synthesis, although the primers may contain more or fewer nucleotides. Shorter primers generally require lower temperatures to form sufficiently stable hybrid complexes with target sequences. The capability of polynucleotides to anneal can be determined by the melting temperature (“Tₘ”) of the hybrid complex. Tₘ is the temperature at which 50% of a polynucleotide strand and its perfect complement form a double-stranded polynucleotide. Therefore, the Tₘ for a selected polynucleotide varies with factors that influence or affect hybridization. In some embodiments, in which thermocycling occurs, the primers can be designed to have a melting temperature (“Tₘ”) in the range of about 60-75° C. Melting temperatures in this range tend to insure that the primers remain annealed or hybridized to the target polynucleotide at the initiation of primer extension. The actual temperature used for a primer extension reaction may depend upon, among other factors, for example, the concentration of the primers. For reactions carried out with a thermostable polymerase such as Taq DNA polymerase, in exemplary embodiments primers can be designed to have a Tₘ in the range of about 60 to about 78° C. or from about 55 to about 70° C. The melting temperatures of the different primers can be different; however, in an alternative embodiment they should all be approximately the same, i.e., the Tₘ of each primer, for example, in a parallel reaction can be within a range of about 5° C. or less. The Tₘs of various primers can be determined empirically utilizing melting techniques that are well-known in the art (see, e.g., Sambrook et al. Molecular Cloning: A Laboratory Manual 11.55-11.57 (2d. ed., Cold Spring Harbor Laboratory Press)). Alternatively, the Tₘ of a primer can be calculated. Numerous references and aids for calculating Tₘₛ of primers are available in the art and include, by way of example and not limitation, Baldino et al. Methods Enzymology. 168:761-777; Bolton et al., 1962, Proc. Natl. Acad. Sci. USA 48:1390; Bresslauer et al., 1986, Proc. Natl. Acad. Sci. USA 83:8893-8897; Freier et al., 1986, Proc. Natl. Acad. Sci. USA 83:9373-9377; Kierzek et al., Biochemistry 25:7840-7846; Montpetit et al., 1992, J. Virol. Methods 36:119-128; Osborne, 1991, CABIOS 8:83; Rychlik et al., 1990, Nucleic Acids Res. 18:6409-6412 (erratum, 1991, Nucleic Acids Res. 19:698); Rychlik. J. NIH Res. 6:78; Sambrook et al. Molecular Cloning: A Laboratory Manual 9.50-9.51, 11.46-11.49 (2d. ed., Cold Spring Harbor Laboratory Press); Sambrook et al., Molecular Cloning: A Laboratory Manual 10.1-10.10 (3d. ed. Cold Spring Harbor Laboratory Press)); SantaLucia, 1998, Proc. Natl. Acad. Sci. USA 95:1460-1465; Suggs et al., 1981, In Developmental Biology Using Purified Genes (Brown et al., eds.), pp. 683-693, Academic Press; Wetmur, 1991, Crit. Rev. Biochem. Mol. Biol. 26:227-259, which disclosures are incorporated by reference. Any of these methods can be used to determine a Tₘ of a primer.

As the skilled artisan will appreciate, in general, the relative stability and therefore, the Tₘs, of RNA:RNA, RNA:DNA, and DNA:DNA hybrids having identical sequences for each strand may differ. In general, RNA:RNA hybrids are the most stable (highest relative Tₘ) and DNA:DNA hybrids are the least stable (lowest relative Tₘ). Accordingly, in some embodiments, another factor to consider, in addition to those described above, when designing a primer is the structure of the primer and target polynucleotide. For example, in the embodiment in which an RNA polynucleotide is reverse transcribed to produce a cDNA, the determination of the suitability of a DNA primer for the reverse transcription reaction should include the effect of the RNA polynucleotide on the Tₘ of the primer. Although the Tₘs of various hybrids may be determined empirically, as described above, examples of methods of calculating the Tₘ of various hybrids are found at Sambrook et al. Molecular Cloning: A Laboratory Manual 9.51 (2d. ed., Cold Spring Harbor Laboratory Press).

The sequences of primers useful for the disclosed methods are designed to be substantially complementary to regions of the target polynucleotides. By “substantially complementary” herein is meant that the sequences of the primers include enough complementarity to hybridize to the target polynucleotides at the concentration and under the temperature and conditions employed in the reaction and to be extended by the DNA polymerase.

In some embodiments, primers can be a nucleobase polymer. By “nucleobase” is meant naturally occurring and synthetic heterocyclic moieties commonly known to those who utilize nucleic acid or polynucleotide technology or utilize polyamide or peptide nucleic acid technology to generate polymers that can hybridize to polynucleotides in a sequence-specific manner. Non-limiting examples of suitable nucleobases include: adenine, cytosine, guanine, thymine, uracil, 5-propynyl-uracil, 2-thio-5-propynyl-uracil, 5-methylcytosine, pseudoisocytosine, 2-thiouracil and 2-thiothymine, 2-aminopurine, N9-(2-amino-6-chloropurine), N9-(2,6-diaminopurine), hypoxanthine, N9-(7-deaza-guanine), N9-(7-deaza-8-aza-guanine) and N8-(7-deaza-8-aza-adenine). Other non-limiting examples of suitable nucleobases include those nucleobases disclosed in FIGS. 2(A) and 2(B) of Buchardt et al. (U.S. Pat. No. 6,357,163; WO 92/20702 and WO 92/20703).

The skilled artisan will appreciate that the suitability of any nucleobase used in a primer can depend, at least in part, on the intended use of the primer. For example, a nucleobase suitable for a sequencing primer may not be suitable as a multiplex amplification or clonal amplification primer. This is because particular nucleobases may not provide a suitable template for a polymerase. For example, peptide-nucleic acids (PNAs), described below, do not provide a suitable template for polymerases. Therefore, primers comprising one or more PNAs, are generally, not suitable for exponential amplifications by PCR because DNA synthesis ceases when a thermostable polymerase encounters the PNA in the template strand. However, primers comprising PNA can be suitable for sequencing reactions and amplification reactions that do not require a polymerase to read through the PNA, including but not limited to, linear PCR amplifications. Determining the types of nucleobases suitable for primers employed in the various types of amplification and analysis reactions as disclosed herein is within the abilities of the skilled artisan.

Nucleobases can be linked to other moieties to form nucleosides, nucleotides, and nucleoside/tide analogs. As used herein, “nucleoside” refers to a compound consisting of a purine, deazapurine, or pyrimidine nucleoside base, e.g., adenine, guanine, cytosine, uracil, thymine, 7-deazaadenine, 7-deazaguanosine, that is linked to the anomeric carbon of a pentose sugar at the 1′ position, such as a ribose, 2′-deoxyribose, or a 2′,3′-di-deoxyribose. When the nucleoside base is purine or 7-deazapurine, the pentose is attached at the 9-position of the purine or deazapurine, and when the nucleoside base is pyrimidine, the pentose is attached at the 1-position of the pyrimidine (see, e.g., Kornberg and Baker, DNA Replication, 2nd Ed. (Freeman 1992)). The term “nucleotide” as used herein refers to a phosphate ester of a nucleoside, e.g., a mono-, a di-, or a triphosphate ester, wherein the most common site of esterification is the hydroxyl group attached to the C-5 position of the pentose. “Nucleotide 5′-triphosphate” refers to a nucleotide with a triphosphate ester group at the 5′ position. The term “nucleoside/tide” as used herein refers to a set of compounds including both nucleosides and/or nucleotides.

“Nucleobase polymer or oligomer” refers to two or more nucleobases connected by linkages that permit the resultant nucleobase polymer or oligomer to hybridize to a polynucleotide having a complementary nucleobase sequence. Nucleobase polymers or oligomers include, but are not limited to, poly- and oligonucleotides (e.g., DNA and RNA polymers and oligomers), poly- and oligonucleotide analogs and poly- and oligonucleotide mimics, such as polyamide or peptide nucleic acids. Nucleobase polymers or oligomers can vary in size from a few nucleobases, from 2 to 40 nucleobases, to several hundred nucleobases, to several thousand nucleobases, or more.

“Polynucleotide or oligonucleotide” refers to nucleobase polymers or oligomers in which the nucleobases are connected by sugar phosphate linkages (sugar-phosphate backbone). Exemplary poly- and oligonucleotides include polymers of 2′-deoxyribonucleotides (DNA) and polymers of ribonucleotides (RNA). A polynucleotide may be composed entirely of ribonucleotides, entirely of 2′-deoxyribonucleotides or combinations thereof.

In some embodiments, a nucleobase polymer is an polynucleotide analog or an oligonucleotide analog. By “polynucleotide analog or oligonucleotide analog” is meant nucleobase polymers or oligomers in which the nucleobases are connected by a sugar phosphate backbone comprising one or more sugar phosphate analogs. Typical sugar phosphate analogs include, but are not limited to, sugar alkylphosphonates, sugar phosphoramidites, sugar alkyl- or substituted alkylphosphotriesters, sugar phosphorothioates, sugar phosphorodithioates, sugar phosphates and sugar phosphate analogs in which the sugar is other than 2′-deoxyribose or ribose, nucleobase polymers having positively charged sugar-guanidyl interlinkages such as those described in U.S. Pat. No. 6,013,785 and U.S. Pat. No. 5,696,253 (see also, Dagani, 1995, Chem. & Eng. News 4-5:1153; Dempey et al., 1995, J. Am. Chem. Soc. 117:6140-6141). Such positively charged analogues in which the sugar is 2′-deoxyribose are referred to as “DNGs,” whereas those in which the sugar is ribose are referred to as “RNGs.” Specifically included within the definition of poly- and oligonucleotide analogs are locked nucleic acids (LNAs; see, e.g., Elayadi et al., 2002, Biochemistry 41:9973-9981; Koshkin et al., 1998, J. Am. Chem. Soc. 120:13252-3; Koshkin et al., 1998, Tetrahedron Letters, 39:4381-4384; Jumar et al., 1998, Bioorganic & Medicinal Chemistry Letters 8:2219-2222; Singh and Wengel, 1998, Chem. Commun., 12:1247-1248; WO 00/56746; WO 02/28875; and, WO 01/48190.

In some embodiments, a nucleobase polymer is a polynucleotide mimic or oligonucleotide mimic. “Polynucleotide mimic or oligonucleotide mimic” refers to a nucleobase polymer or oligomer in which one or more of the backbone sugar-phosphate linkages is replaced with a sugar-phosphate analog. Such mimics are capable of hybridizing to complementary polynucleotides or oligonucleotides, or polynucleotide or oligonucleotide analogs or to other polynucleotide or oligonucleotide mimics, and may include backbones comprising one or more of the following linkages: positively charged polyamide backbone with alkylamine side chains as described in U.S. Pat. Nos. 5,786,461, 5,766,855, 5,719,262, 5,539,082 and WO 98/03542 (see also, Haaima et al., 1996, Angewandte Chemie Int'l Ed. in English 35:1939-1942; Lesnick et al., 1997, Nucleotid. 16:1775-1779; D'Costa et al., 1999, Org. Lett. 1:1513-1516; Nielsen, 1999, Curr. Opin. Biotechnol. 10:71-75); uncharged polyamide backbones as described in WO 92/20702 and U.S. Pat. No. 5,539,082; uncharged morpholino-phosphoramidate backbones as described in U.S. Pat. Nos. 5,698,685, 5,470,974, 5,378,841, and 5,185,144 (see also, Wages et al., 1997, BioTechniques 23:1116-1121); peptide-based nucleic acid mimic backbones (see, e.g., U.S. Pat. No. 5,698,685); carbamate backbones (see, e.g, Stirchak and Summerton, 1987, J. Org. Chem. 52:4202); amide backbones (see, e.g., Lebreton, 1994, Synlett. February, 1994:137); methylhydroxylamine backbones (see, e.g., Vasseur et al., 1992, J. Am. Chem. Soc. 114:4006); 3′-thioformacetal backbones (see, e.g., Jones et al., 1993, J. Org. Chem. 58:2983) and sulfamate backbones (see, e.g., U.S. Pat. No. 5,470,967). All of the preceding references are herein incorporated by reference.

“Peptide nucleic acid” or “PNA” refers to poly- or oligonucleotide mimics in which the nucleobases are connected by amino linkages (uncharged polyamide backbone) such as described in any one or more of U.S. Pat. Nos. 5,539,082, 5,527,675, 5,623,049, 5,714,331, 5,718,262, 5,736,336, 5,773,571, 5,766,855, 5,786,461, 5,837,459, 5,891,625, 5,972,610, 5,986,053, 6,107,470, 6,451,968, 6,441,130, 6,414,112 and 6,403,763; all of which are incorporated herein by reference. The term “peptide nucleic acid” or “PNA” shall also apply to any oligomer or polymer comprising two or more subunits of those polynucleotide mimics described in the following publications: Lagriffoul et al., 1994, Bioorganic & Medicinal Chemistry Letters, 4:1081-1082; Petersen et al., 1996, Bioorganic & Medicinal Chemistry Letters, 6:793-796; Diderichsen et al., 1996, Tett. Lett. 37:475-478; Fujii et al., 1997, Bioorg. Med. Chem. Lett. 7:637-627; Jordan et al., 1997, Bioorg. Med. Chem. Lett. 7:687-690; Krotz et al., 1995, Tett. Lett. 36:6941-6944; Lagriffoul et al., 1994, Bioorg. Med. Chem. Lett. 4:1081-1082; Diederichsen, 1997, Bioorg. Med. Chem. 25 Letters, 7:1743-1746; Lowe et al., 1997, J. Chem. Soc. Perkin Trans. 1, 1:539-546; Lowe et al., 1997, J. Chem. Soc. Perkin Trans. 11:547-554; Lowe et al., 1997, I. Chem. Soc. Perkin Trans. 1 1:555-560; Howarth et al., 1997, I. Org. Chem. 62:5441-5450; Altmann et al., 1997, Bioorg. Med. Chem. Lett., 7:1119-1122; Diederichsen, 1998, Bioorg. Med. Chem. Lett., 8:165-168; Diederichsen et al., 1998, Angew. Chem. mt. Ed., 37:302-305; Cantin et al., 1997, Tett. Lett., 38:4211-4214; Ciapetti et al., 1997, Tetrahedron, 53:1167-1176; Lagriffoule et al., 1997, Chem. Eur. 1. 3:912-919; Kumar et al., 2001, Organic Letters 3(9):1269-1272; and the Peptide-Based Nucleic Acid Mimics (PENAMs) of Shah et al. as disclosed in WO 96/04000.

Some examples of PNAs are those in which the nucleobases are attached to an N-(2-aminoethyl)-glycine backbone, i.e., a peptide-like, amide-linked unit (see, e.g., U.S. Pat. No. 5,719,262; Buchardt et al., 1992, WO 92/20702; Nielsen et al., 1991, Science 254:1497-1500).

In some embodiments, a nucleobase polymer is a chimeric oligonucleotide. By “chimeric oligonucleotide” is meant a nucleobase polymer or oligomer comprising a plurality of different polynucleotides, polynucleotide analogs and polynucleotide mimics. For example a chimeric oligo may comprise a sequence of DNA linked to a sequence of RNA. Other examples of chimeric oligonucleotides include a sequence of DNA linked to a sequence of PNA, and a sequence of RNA linked to a sequence of PNA.

In some embodiments, various components of the disclosed methods, including but not limited to primers, ddNTPs, and the reaction compartments, can comprise a detectable moiety. “Detectable moiety,” “detection moiety” or “label” refer to a moiety that renders a molecule to which it is attached detectable or identifiable using known detection systems (e.g., spectroscopic, radioactive, enzymatic, chemical, photochemical, biochemical, immunochemical, chromatographic, physical (e.g., sedimentation, centrifugation, density), electrophoretic, gravimetric, or magnetic systems). Non-limiting examples of labels include quantum dots, isotopic labels (e.g., radioactive or heavy isotopes), magnetic labels; spin labels, electric labels; thermal labels; colored labels (e.g., chromophores), luminescent labels (e.g., fluorescers, chemiluminescers), enzyme labels (e.g., horseradish peroxidase, alkaline phosphatase, luciferase, β-galactosidase) (Ichiki, et al., 1993, J. Immunol. 150(12):5408-5417; Nolan, et al., 1988, Proc. Natl. Acad. Sci. USA 85(8):2603-2607)), antibody labels, and chemically modifiable labels. In addition, in some embodiments, such labels include components of ligand-binding partner pairs (e.g., antigen-antibody (including single-chain antibodies and antibody fragments, e.g., FAb, F(ab)′₂, Fab′, Fv, etc. (Fundamental Immunology 47-105 (William E. Paul ed., 5^{th} ed., Lippincott Williams & Wilkins 2003)), hormone-receptor binding, neurotransmitter-receptor binding, polymerase-promoter binding, substrate-enzyme binding, inhibitor-enzyme binding (e.g., sulforhodamine-valyl-alanyl-aspartyl-fluoromethylketone (SR-VAD-FMK-caspase(s) binding), allosteric effector-enzyme binding, biotin-streptavidin binding, digoxin-antidigoxin binding, carbohydrate-lectin binding, Annexin V-phosphatidylserine binding (Andree et al., 1990, J. Biol. Chem. 265(9):4923-8; van Heerde et al., 1995, Thromb. Haemost. 73(2):172-9; Tait et al., 1989, J. Biol. Chem. 264(14):7944-9), nucleic acid annealing or hybridization, or a molecule that donates or accepts a pair of electrons to form a coordinate covalent bond with the central metal atom of a coordination complex. In various exemplary embodiments the dissociation constant of the binding ligand can be less than about 10⁻⁴-10⁻⁶ M⁻¹, less than about 10⁻⁵ to 10⁻⁹ M⁻¹, or less than about 10⁻⁷-10⁻⁹ M⁻¹.

“Fluorescent label,” “fluorescent moiety,” and “fluorophore” refer to a molecule that may be detected via its inherent fluorescent properties. Examples of suitable fluorescent labels include, but are not limited to, fluorescein, rhodamine, tetramethylrhodamine, eosin, erythrosin, coumarin, methyl-coumarins, pyrene, Malachite Green, stilbene, Lucifer Yellow, Cascade BlueJ, Texas Red, IAEDANS, EDANS, BODIPY FL, LC Red 640, phycoerythrin, LC Red 705, Oregon green, Alexa-Fluor dyes (Alexa Fluor 350, Alexa Fluor 430, Alexa Fluor 488, Alexa Fluor 546, Alexa Fluor 568, Alexa Fluor 594, Alexa Fluor 633, Alexa Fluor 660, Alexa Fluor 680), Cascade Blue, Cascade Yellow and R-phycoerythrin (PE), FITC, Rhodamine, Texas Red (Pierce, Rockford, Ill.), Cy5, Cy5.5, Cy7 (Amersham Life Science, Pittsburgh, Pa.) and tandem conjugates, such as but not limited to, Cy5PE, Cy5.5PE, Cy7PE, Cy5.5APC, Cy7APC. In some embodiments, suitable fluorescent labels also include, but are not limited to, green fluorescent protein (GFP; Chalfie, et al., 1994, Science 263(5148):802-805), EGFP (Clontech Laboratories, Inc., Palo Alto, Calif.), blue fluorescent protein (BFP; Quantum Biotechnologies, Inc. Montreal, Canada; Heim et al, 1996, Curr. Biol. 6:178-182; Stauber, 1998, Biotechniques 24(3):462-471;), enhanced yellow fluorescent protein (EYFP; Clontech Laboratories, Inc., Palo Alto, Calif.), and renilla (WO 92/15673; WO 95/07463; WO 98/14605; WO 98/26277; WO 99/49019; U.S. Pat. Nos. 5,292,658, 5,418,155, 5,683,888, 5,741,668, 5,777,079, 5,804,387, 5,874,304, 5,876,995, and 5,925,558). Further examples of fluorescent labels are found in Haugland, Handbook of Fluorescent Probes and Research, 9^{th} Edition, Molecule Probes, Inc. Eugene, Oreg. (ISBN 0-9710636-0-5).

In some embodiments, a label can be a microparticle. By “microparticle”, “microsphere”, “microbead”, “bead” and grammatical equivalents herein are meant a small discrete synthetic particle. As known in the art, the composition of beads can vary depending on the type of assay in which they are used and, therefore, selecting a microbead composition is within the abilities of the practitioner. Suitable bead compositions include those used in peptide, nucleic acid and organic synthesis, including, but not limited to, plastics, ceramics, glass, polystyrene, methylstyrene, acrylic polymers, paramagnetic materials (U.S. Pat. Nos. 4,358,388, 4,654,267, 4,774,265, 5,320,944, 5,356,713), thoria sol, carbon graphite, titanium dioxide, latex or cross-linked dextrans such as Sepharose, agarose, cellulose, carboxymethyl cellulose, hydroxyethyl cellulose, proteinaceous polymer, nylon, globulin, DNA, cross-linked micelles and Teflon may all be used (see, e.g., Microsphere Detection Guide from Bangs Laboratories, Fishers, Ind.), Beads are also commercially available from, for example, Bio-Rad Laboratories (Richmond, Calif.), LKB (Sweden), Pharmacia (Piscataway, N.J.), IBF (France), Dynal Inc. (Great Neck, N.Y.). In some embodiments, beads may contain a cross-linking agent, such as, but not limited to divinyl benzene, ethylene glycol dimethacrylate, trimethylol propane trimethacrylate, N,N′methylene-bis-acrylamide, adipic acid, sebacic acid, succinic acid, citric acid, 1,2,3,4-butanetetracarboxylic acid, or 1,10 decanedicarboxylic acid or other functionally equivalent agents known in the art. In various exemplary embodiments, beads can be spherical, non-spherical, egg-shaped, irregularly shaped, and the like. The average diameter of a microparticle can be selected at the discretion of the practitioner. However, generally the average diameter of microparticle can range from nanometers (e.g. about 100 nm) to millimeters (e.g. about 1 mm) with beads from about 0.2 μm to about 200 μm being preferred, and from about 0.5 to about 10 μm being particularly preferred, although in some embodiments smaller or larger beads may be used, as described below.

In some embodiments a microparticle can be porous, thus increasing the surface area available for attachment to another molecule, moiety, or compound (e.g., a primer). Thus, microparticles may have additional surface functional groups to facilitate attachment and/or bonding. These groups may include carboxylates, esters, alcohols, carbamides, aldehydes, amines, sulfur oxides, nitrogen oxides, or halides. Methods of attaching another molecule or moiety to a bead are known in the art (see, e.g., U.S. Pat. Nos. 6,268,222, 6,649,414). In some embodiments, a microparticle can further comprise a label.

The compositions and reagents described herein can be packaged into kits. In some embodiments, a kit comprises a reagent for making an inverse emulsion comprising one or more aqueous compartments. In some embodiments, the aqueous compartments can be used in conjunction with one or more reagents from commercially available kits, including, but not limited to, those available from Applied Biosystems (i.e., Big Dye® Terminator Cycle Sequencing Kit), Epicentre (i.e., SequiTherm™ Cycle Sequencing Kit), Amersham (i.e., DYEnamic Direct Dye-Primer Cycle Sequencing Kits), Boehringer Mannheim (i.e., CycleReader™ DNA Sequencing Kit), Bionexus Inc. (i.e., AccuPower DNA Sequencing Kit), and USB cycle sequencing kits (i.e., Thermo Sequenase™ Cycle Sequencing Kit).

In some embodiments, a kit can comprise a primer suitable for multiplex or clonal amplification. In some embodiments, a primer can be attached to a surface, such as, a microparticle and/or a slide and the like. In some embodiments each primer can comprise a target specific sequence and/or a universal sequence. In some embodiments, the microparticles can further comprise various labels, including but not limited to, fluorescent and/or magnetic labels. In some embodiments, a kit can comprise a library of primers or primer pairs. In some embodiments, a kit can comprise one or more reaction compartments comprising reagents suitable for performing a reaction selected at the discretion of a practitioner. For example, in some embodiments, a kit can comprise one or more reaction compartments comprising one more sequencing reagents.

The various components included in the kit are typically contained in separate containers, however, in some embodiments, one or more of the components can be present in the same container. Additionally, kits can comprise any combination of the compositions and reagents described herein. In some embodiments, kits can comprise additional reagents that may be necessary or optional for performing the disclosed methods. Such reagents include, but are not limited to, buffers, molecular size standards, control polynucleotides, and the like.

In this application, the use of the singular includes the plural unless specifically stated otherwise. The section headings used herein are for organizational purposes only and are not to be construed as limiting the subject matter described in any way. While the present teachings are described in conjunction with various embodiments, it is not intended that the present teachings be limited to such embodiments. On the contrary, the present teachings encompass various alternatives, modifications, and equivalents, as will be appreciated by those of skill in the art.

All literature and similar materials cited in this application, including but not limited to, patents, patent applications, articles, books, and treatises, regardless of the format of such literature and similar materials, are expressly incorporated by reference in their entirety for any purpose. In the event that one or more of the incorporated literature and similar materials differs from or contradicts this application, including but not limited to defined terms, term usage, described techniques, or the like, this application controls.

1. A method of analyzing a plurality of polynucleotides comprising:
a) amplifying a plurality of polynucleotides under conditions suitable to produce a plurality of multiplex amplicons;
b) clonally amplifying said multiplex amplicons to produce a plurality of clonal amplicons; and
c) analyzing said plurality of clonal amplicons.

2. The method according to claim 1, wherein said plurality of polynucleotides is at least about 100 polynucleotides.

3. The method according to claim 1, wherein said plurality of polynucleotides is at least about 1000 polynucleotides.

4. The method according to claim 1, wherein said plurality of polynucleotides is at least about 10,000 polynucleotides.

5. The method according to claim 1, wherein said plurality of polynucleotides is at least about 100,000 polynucleotides.

6. The method according to claim 1, wherein said plurality of polynucleotides is at least about 1,000,000 polynucleotides.

7. (canceled)

8. (canceled)

9. The method according to claim 1, wherein said multiplex amplicons are clonally amplified in hydrophilic compartments of an inverse emulsion.

10. The method according to claim 9, wherein said hydrophilic compartments are disposed upon a surface.

11. The method according to claim 10, wherein said surface comprises primers suitable for clonally amplifying said multiplex amplicons.

12. The method according to claim 11, wherein said primers are hybridized to said multiplex amplicons.

13. The method according to claim 12, wherein said clonal amplicons are attached to said surface.

14. The method according to claim 1, wherein said conditions suitable for producing said plurality of multiplex amplicons comprise multiple rounds of a thermocycling reaction comprising forward and reverse amplification primer pairs, a thermostable polymerase, and deoxynucleotide triphosphate suitable for DNA synthesis.

15. The method according to claim 14, wherein said multiple rounds of a thermocycling reaction terminates before said reaction reaches a plateau.

16. The method according to claim 14, wherein said forward primers comprises a forward universal sequence and said reverse primers comprise a reverse universal sequence.

17. (canceled)

18. (canceled)

19. (canceled)

20. (canceled)

21. (canceled)

22. The method according to claim 1, wherein said analyzing comprises sequencing said plurality of clonal amplicons.

23. The method according to claim 22, wherein said sequencing comprising sequencing in parallel.

24. The method according to claim 22, wherein said sequencing is massively parallel signature sequence.

25. (canceled)

26. (canceled)

27. (canceled)

28. A method of amplifying a plurality of polynucleotides comprising: hybridizing polynucleotides isolated in hydrophilic compartments of an inverse emulsion to primers, wherein said primers are attached to a surface and said compartments are disposed upon said surface, and wherein said polynucleotides and said primers are hybridized in the presence of a polymerase and nucleotide triphosphates suitable for nucleic acid synthesis and under conditions suitable for said polymerase to extend said primers, whereby said polynucleotides are amplified.

29. (canceled)

30. (canceled)

31. (canceled)

32. (canceled)

33. (canceled)

34. (canceled)

35. A method of analyzing a plurality of polynucleotides comprising:
a) clonally amplifying a plurality of polynucleotides in hydrophilic compartments of an inverse emulsion in the presence of primers, a polymerase, and nucleotide triphosphates suitable for nucleic acid synthesis, wherein said primer is attached to a surface and said compartment is disposed upon said surface, and under conditions suitable to produce a plurality of clonal amplicons attached to said surface; and
b) analyzing said plurality of clonal amplicons.

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. A composition comprising an inverse emulsion and an oligonucleotide attached to a surface, wherein said inverse emulsion comprises at least one hydrophilic compartment that is disposed upon a said surface and wherein said oligonucleotide is in contact with said hydrophilic compartment.

52. (canceled)
